SEQUENCE LISTING (2 sequences)

SEQ ID NO: 1 - DNA, 1668 bp, Bacillus subtilis
agctcggatccactagtaacggccgccagtgtgctggaattcgcccttagaaaggaggtgatccagccgcaccttccgatacggctaccttgttacgacttcaccccaatcatctgtcccaccttcggcggctggctccataaaggttacctcaccgacttcgggtgttacaaactctcgtggtgtgacgggcggtgtgtacaaggcccgggaacgtattcaccgcggcatgctgatccgcgattactagcgattccagcttcacgcagtcgagttgcagactgcgatccgaactgagaacagatttgtgrgattggcttaacctcgcggtttcgctgccctttgttctgtccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgcccccgaaggggacgtcctatctctaggattgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaaggggcggaaaccccctaacacttagcactcatcgtttacggcgtggactaccagggtatctaatcctgttcgctccccacgctttcgctcctcagcgtcagttacagaccagagagtcgccttcgccactggtgttcctccacatctctacgcatttcaccgctacacgtggaattccactctcctcttctgcactcaagttccccagtttccaatgaccctccccggttgagccgggggctttcacatcagacttaagaaaccgcctgcgagccctttacgcccaataattccggacaacgcttgccacctacgtattaccgcggctgctggcacgtagttagccgtggctttctggttaggtaccgtcaaggtgccgccctatttgaacggcacttgttcttccctaacaacagagctttacgatccgaaaaccttcatcactcacgcggcgttgctccgtcagactttcgtccattgcggaagattccctactgctgcctcccgtaggagtctgggccgtgtctcagtcccagtgtggccgatcaccctctcaggtcggctacgcatcgtcgccttggtgagccgttacctcaccaactagctaatgcgccgcgggtccatctgtaagtggtagccgaagccaccttttatgtctgaaccatgcggttcagacaaccatccggtattagccccggtttcccggagttatcccagtcttacaggcaggttacccacgtgttactcacccgtccgccgctaacatcagggagcaagctcccatctgtccgctcgacttgcatgtattaggcacgccgccagcgttcgtcctgagccatgaacaaactctaagggcgaattctgcagatatccatcacactggcggccgctcgagcatgcatctagagggcccaatcgccctat

SEQ ID NO: 2 - DNA, 483 bp, Artificial Sequence
  Artificial polynucleotide
ttgaacgctggcggcatgccttacacatgcaagtcgaacggtaacgcggggcaacctggcgacgagtggcgaacgggtgagtaatgtatcggaacgtgcccagttgtgggggataactgctcgaaagagcagctaataccgcatacgacctgagggtgaaagcgggggatcgcaagacctcgcgcaattggagcggccgatatcagattaggtagttggtggggtaaaggcctaccaagccgacgatctgtagctggtctgagaggacgaccagccacactgggactgagacacggcccagactcctacgggaggcagcagtggggaattttggacaatgggggcaaccctgatccagccatgccgcgtgcgggaagaaggccttcgggttgtaaaccgcttttgtcagggaagaaaagactcctactaatactgggggttcatgacggtacctgaagaataagcaccggctaactacgtgcc

(19) United States
(12) Patent Application Publication
(10) Pub. No.: US 2019/0119134 A1
(43) Pub. Date: Apr. 25, 2019

(54) COMPOSITIONS AND METHODS FOR REDUCING CYANURIC ACID IN RECREATIONAL WATER SYSTEMS

(71) Applicant: BiOWiSH Technologies Inc., Cincinnati, OH (US)

(72) Inventors: John GORSUCH, Cincinnati, OH (US); Michael Stanford SHOWELL, Cincinnati, OH (US)

(21) Appl. No.: 16/166,963

(22) Filed: Oct. 22, 2018

Related U.S. Application Data
(60) Provisional application No. 62/625,034, filed on Feb. 1, 2018.
(60) Provisional application No. 62/575,148, filed on Oct. 20, 2017.

(51) Int. Cl. C02F 3/34; C02F 3/10; C12N 1/20

(52) U.S. Cl. CPC C02F 3/341; C02F 3/348; C02F 2101/38; C12N 1/20; C02F 2305/06; C02F 3/10

(57) ABSTRACT

The present invention provides compositions and methods of reducing cyanuric acid levels in recreational water systems.

### CROSS-REFERENCE TO RELATED APPLICATIONS

This application claims the benefit of and priority to U.S. Provisional Application No. 62/575,148, filed on Oct. 20, 2017, and U.S. Provisional Application No. 62/625,034, filed on Feb. 1, 2018, the contents of each of which are hereby incorporated by reference in their entireties.

### INCORPORATION BY REFERENCE OF SEQUENCE LISTING

The contents of the text file named “BIOW-015_001US_SEQ_LISTING.txt”, which was created on Sep. 18, 2018 and is 3.33 KB in size, are hereby incorporated by reference in their entireties.

### FIELD OF THE INVENTION

The present invention relates to cyanuric acid reduction in water systems, particularly recreational water systems such as swimming pools.

### BACKGROUND OF THE INVENTION

Recreational water systems such as swimming pools, spas, hot tubs, and jetted tubs, are commonly treated with chlorinated derivatives of cyanuric acid (1,3,5-triazine-2,4,6(1H,3H,5H)-trione) in order to disinfect the water and maintain sanitary conditions. The action of these chlorinated cyanuric acid derivatives, typically referred to by the trade names di- or trichlor, is attributed to the generation of free chlorine as HOCl and OCl⁻ arising from the hydrolytic equilibria of the various chlorinated species. When used in this way there is a gradual accumulation of residual cyanuric acid in the water. As the level of cyanuric acid rises, free chlorine's ability to act as a disinfectant is weakened due to increased complexation of chlorine. Above about 50 ppm cyanuric acid, the time it takes to kill bacteria in chlorinated water increases versus similarly treated water without cyanuric acid. In heated systems, such as hot tubs and spas, at even moderate levels of cyanuric acid the amount of time it takes chlorine to kill a common pathogen such as pseudomonas aeruginosa can be as much as one hundred times as long as similar systems without cyanuric acid.

A 2007 study by the United States Centers for Disease Control and Prevention revealed that cyanuric acid significantly diminishes chlorine's ability to inactivate chlorine-resistant porotozoan and cryptosporidium. Based on these findings several state and local Departments of Health have issued recommendations to the recreational water industry that cyanuric acid levels not exceed 30 ppm.

It is a common practice in the recreational water industry to reduce excess cyanuric acid levels by partially draining pools, tubs, spas, holding tanks, etc., and refilling with fresh water. This is a labor intensive and costly solution, particularly in areas affected by prolonged drought such as Southern California where the cost to replenish a typical 20,000 gallon swimming pool with fresh water is prohibitively high. Accordingly, a need exists in the recreational water industry for compositions and methods to reduce excess cyanuric acid levels that do not require draining and replenishing.

### SUMMARY OF THE INVENTION

The present disclosure provides compositions and methods for augmenting the treatment of commercial, public, and private recreational water systems such as swimming pools, spas, hot tubs, jetted tubs or the like. The compositions and methods can result in decreased cyanuric acid levels in the water. In specific embodiments, the compositions and methods are used to reduce cyanuric acid levels in recreational water systems where cyanuric acid stabilized chlorine is used as part of the routine disinfection and sanitization protocol.

In one aspect, the present disclosure provides a filter assembly comprising: a solid support; a biofilm comprising (a) an organism having a 16S sequence at least 90% identical to SEQ ID NO: 2 and Bacillus subtilis 34 KLB, (b) Enterobacter cloacae and Bacillus subtilis 34 KLB, (c) Bacillus subterraneous and Bacillus subtilis 34 KLB, or (d) Bacillus subtilis 34 KLB and two or more organisms selected from Enterobacter cloacae, Bacillus subterraneous, and an organism having a 16S sequence at least 90% identical to SEQ ID NO: 2, wherein the biofilm is disposed on the solid support; and a porous filter housing, wherein the solid support is encased in the porous filter housing.

In some embodiments, the solid support is porous.

In some embodiments, the solid support comprises zeolite, wheat bran, rice bran, ground corn cobs, bentonite, kaolin, diatomaceous earth, activated charcoal, calcium carbonate, calcium pyrophosphate, tri-calcium phosphate, sphagnum moss, glass, sand, cellulose, ceramic, polyethylene, polypropylene, polystyrene, uncooked starch, or a mixture thereof.

In some embodiments, the porous filter housing has an average pore size in the range of 0.2 μm to 1.0 μm (e.g., about 0.5 μm).

In some embodiments, the filter assembly is configured to be used in a swimming pool filtration system.

In some embodiments, the biofilm is in the form of a pellet or tablet.

In another aspect, the present disclosure provides a kit comprising the filter assembly described herein and a bacterial composition comprising: (a) a mixture of Bacillus bacterial species comprising Bacillus subtilis, Bacillus mojavensis, Bacillus licheniformis, Bacillus amyloliquefaciens, and Bacillus pumilus; and (b) a mixture of lactic-acid-producing bacterial species comprising Pediococcus acidilactici, Pediococcus pentosaceus, and Lactobacillus plantarum. In some embodiments, the Bacillus subtilis comprises Bacillus subtilis 34 KLB.

In another aspect, the present disclosure provides a kit comprising the filter assembly described herein and a bacterial composition comprising: (a) a mixture of Bacillus bacterial species comprising Bacillus subtilis, Bacillus subtilis 34 KLB, Bacillus licheniformis, Bacillus amyloliquefaciens, and Bacillus pumilus; and (b) a mixture of lactic-acid-producing bacterial species comprising Pediococcus acidilactici, Pediococcus pentosaceus, and Lactobacillus plantarum.

In another aspect, the present disclosure provides a method for reducing the concentration of cyanuric acid in a water system, comprising: contacting the water system with the filter assembly described herein; and contacting the water system with a bacterial composition, wherein the bacterial composition comprises: (a) between 75-99% w/w of a water-soluble or water-dispersible carbon source; (b) a mixture of Bacillus bacterial species comprising Bacillus subtilis, Bacillus subtilis 34 KLB, Bacillus licheniformis, Bacillus amyloliquefaciens, and Bacillus pumilus; and (c) a mixture of lactic-acid-producing bacterial species comprising Pediococcus acidilactici, Pediococcus pentosaceus, and Lactobacillus plantarum.

In another aspect, the present disclosure provides a method for reducing the concentration of cyanuric acid in a water system, comprising: contacting the water system with the filter assembly of the present disclosure; and contacting the water system with a bacterial composition, wherein the bacterial composition comprises: (a) between 75-99% w/w of a water-soluble or water-dispersible carbon source; (b) a mixture of Bacillus bacterial species comprising Bacillus subtilis, Bacillus mojavensis, Bacillus licheniformis, Bacillus amyloliquefaciens, and Bacillus pumilus; and (c) a mixture of lactic-acid-producing bacterial species comprising Pediococcus acidilactici, Pediococcus pentosaceus, and Lactobacillus plantarum.

In some embodiments, the water system is filtered through the filter assembly.

In some embodiments, the bacterial species in the bacterial composition are non-pathogenic.

In some embodiments, at least 15% of the Bacillus bacterial species in the bacterial composition are Bacillus subtilis 34 KLB.

In some embodiments, each of the lactic-acid-producing bacterial species in the bacterial composition is present in equal amounts by weight.

In some embodiments, the bacterial composition further comprises an inorganic mineral that stimulates bacterial respiration and growth. For example, the inorganic mineral can be selected from the group consisting of disodium hydrogen phosphate, dipotassium hydrogen phosphate, sodium dihydrogen phosphate, potassium dihydrogen phosphate, sodium chloride, potassium chloride, magnesium sulfate, calcium sulfate, magnesium chloride, calcium chloride, and iron(III) chloride.

In some embodiments, the method can further comprise placing the filter assembly into a filtration system in connection with the water system.

In some embodiments, the water system is a swimming pool.

In some embodiments, the water-soluble or water-dispersible carbon source is selected from the group consisting of acetate, succinate, dextrose, sucrose, fructose, erythrose, arabinose, ribose, deoxyribose, galactose, mannose, lactose, maltose, dextrin, maltodextrin, glycerol, sorbitol, xylitol, inulin, trehalose, starch, cellobiose, and carboxy methyl cellulose.

In some embodiments, the dextrose is dextrose monohydrate.

In some embodiments, the bacterial composition has a bacterial concentration of about 0.01 to 10 ppm.

In some embodiments, the biofilm of the filter assembly has a bacterial concentration of about 0.01 to 10 ppm.

In some embodiments, the Bacillus subtilis comprises Bacillus subtilis 34 KLB.

In some embodiments, the mixture of Bacillus bacterial species has a bacterial concentration of at least 1×10⁶ colony forming units (CFU) per gram of the mixture, wherein each of the Bacillus species are individually fermented aerobically, dried and ground to an average particle size of about 200 microns.

In some embodiments, the mixture of lactic-acid-producing bacterial species has a bacterial concentration of at least 1×10⁶ colony forming units (CFU) per gram of the mixture, wherein each of the lactic-acid-producing bacterial species are fermented anaerobically, dried, and ground to an average particle size of about 200 microns.

In some embodiments, the concentration of cyanuric acid in the water system can be reduced by at least 10% by using the methods of the present disclosure.

In some embodiments, the concentration of cyanuric acid in the water system can be reduced by at least 50% by using the methods of the present disclosure.

### BRIEF DESCRIPTION OF THE DRAWINGS

FIG. 1 is a set of photographs of AquaCheck7 test strips showing reduction of cyanuric acid by solid-supported bacteria.

FIG. 2 is a graph showing the synergy of Composition A and Composition C from Example 2 in degrading cyanuric acid.

FIG. 3 is a set of photographs of AquaCheck7 test strips showing reduction of cyanuric acid according to the methods of the present disclosure.

FIG. 4 is a flow chart showing the steps in testing the effectiveness of the system in reducing cyanuric acid. In step 1, fermentation of stock bacterial strains to ensure upregulation of naturally-occurring genes to produce the desired phenotype. In step 2, addition of bacterial assemblage (at desired ratios) to a biofilm-growth system utilizing a solid support substrate and rich medium containing the targeted chemical substrate. Allow biofilm to grow for about 48 hours. In step 3, deploy filter medium in a pool microcosm system and monitor the system for changes in cyanuric acid concentration for about 7 days.

FIG. 5 is a graph showing that Enterobacter cloacae is a robust cyanuric acid reducer.

FIG. 6 is a photograph showing the clearing of cyanuric acid on an agar by an organism having a 16S sequence at least 90% identical to SEQ ID NO: 2. The white flakes are precipitated cyanuric acid. The yellow dot is the original bacterial colony.

### DETAILED DESCRIPTION OF THE INVENTION

The present invention is based upon the surprising discovery that a combination of a water-soluble or water-dispersible carbon source, a mixture of non-pathogenic bacteria and cyanuric acid degrading bacteria has been found to significantly and consistently reduce cyanuric acid levels in recreational waters systems. Recreational water systems include for example a swimming pool, a spa, a hot tub, a jetted tub, or the like.

The non-pathogenic bacteria are derived, for example, from the genus Bacillus, Lactobacillus, Pseudomonas, Enterobacter, or Morella.

Cyanuric acid reducing bacteria include for example Enterobacter cloacae, Bacillus subterraneous, or an organism having a 16S sequence at least 90% identical to SEQ ID NO: 2.

(SEQ ID NO: 2)
TTGAACGCTGGCGGCATGCCTTACACATGCAAGTCGAACGGTAACGCGGG
GCAACCTGGCGACGAGTGGCGAACGGGTGAGTAATGTATCGGAACGTGCC
CAGTTGTGGGGGATAACTGCTCGAAAGAGCAGCTAATACCGCATACGACC
TGAGGGTGAAAGCGGGGGATCGCAAGACCTCGCGCAATTGGAGCGGCCGA
TATCAGATTAGGTAGTTGGTGGGGTAAAGGCCTACCAAGCCGACGATCTG
TAGCTGGTCTGAGAGGACGACCAGCCACACTGGGACTGAGACACGGCCCA
GACTCCTACGGGAGGCAGCAGTGGGGAATTTTGGACAATGGGGGCAACCC
TGATCCAGCCATGCCGCGTGCGGGAAGAAGGCCTTCGGGTTGTAAACCGC
TTTTGTCAGGGAAGAAAAGACTCCTACTAATACTGGGGGTTCATGACGGT
ACCTGAAGAATAAGCACCGGCTAACTACGTGCC.

16S ribosomal RNA (or 16S rRNA) is the component of the 30S small subunit of a prokaryotic ribosome that binds to the Shine-Dalgarno sequence. The 16S rRNA gene is used for phylogenetic studies as it is highly conserved between different species of bacteria and archaea.

In some embodiments, the organism has a 16S sequence at least 91% identical to SEQ ID NO: 2. In some embodiments, the organism has a 16S sequence at least 92% identical to SEQ ID NO: 2. In some embodiments, the organism has a 16S sequence at least 93% identical to SEQ ID NO: 2. In some embodiments, the organism has a 16S sequence at least 94% identical to SEQ ID NO: 2. In some embodiments, the organism has a 16S sequence at least 95% identical to SEQ ID NO: 2. In some embodiments, the organism has a 16S sequence at least 96% identical to SEQ ID NO: 2. In some embodiments, the organism has a 16S sequence at least 97% identical to SEQ ID NO: 2. In some embodiments, the organism has a 16S sequence at least 98% identical to SEQ ID NO: 2. In some embodiments, the organism has a 16S sequence at least 99% identical to SEQ ID NO: 2.

The term “16S organism” is used herein to refer to an organism having a 16S sequence at least 90% identical to SEQ ID NO: 2. As such, the term “16S organism” encompasses an organism having a 16S sequence at least 91% identical to SEQ ID NO: 2, an organism having a 16S sequence at least 92% identical to SEQ ID NO: 2, an organism having a 16S sequence at least 93% identical to SEQ ID NO: 2, an organism having a 16S sequence at least 94% identical to SEQ ID NO: 2, an organism having a 16S sequence at least 95% identical to SEQ ID NO: 2, an organism having a 16S sequence at least 96% identical to SEQ ID NO: 2, an organism having a 16S sequence at least 97% identical to SEQ ID NO: 2, an organism having a 16S sequence at least 98% identical to SEQ ID NO: 2, or an organism having a 16S sequence at least 99% identical to SEQ ID NO: 2.

The 16S organism can be used alone or in combination with one or more other bacterial species to reduce the level of cyanuric acid in a recreational waters system. For example, the 16S organism can be used in combination with Bacillus subtilis 34KLB.

Accordingly, various aspects of the present disclosure provide a composition including a water-soluble or water-dispersible carbon source and a mixture of non-pathogenic bacteria. These compositions are referred to herein as “NPB compositions.”

Additionally, the present disclosure provides a composition including cyanuric acid degrading bacteria on a solid support. These compositions are referred to herein as “CAD compositions.”

The invention also provides methods of treating a recreational water system by contacting the water system with the NPB composition and the CAD composition according to the invention.

The NPB compositions can be in a dried form such a powder, a tablet, a pellet, or a granule. Alternatively, the NPB compositions can be in a liquid form.

In some embodiments, the NPB compositions include a mixture of Bacillus bacterial species and a mixture of lactic-acid-producing bacterial species.

The mixture of Bacillus bacterial species can include one to seven different strains of Bacillus. Exemplary Bacillus bacterial species include Bacillus subtilis, Bacillus amyloliquefaciens, Bacillus licheniformis, Bacillus pumilus, Bacillus megaterium, Bacillus coagulans, Bacillus subterraneous, Bacillus mojavensis, or Paenibacillus polymyxa.

In some embodiments, Bacillus bacterial species include Bacillus subtilis, Bacillus licheniformis, Bacillus amyloliquefaciens, Bacillus mojavensis, and Bacillus pumilus.

In some embodiments, the Bacillus is the Bacillus subtilis strain 34KLB (SEQ ID NO: 1):

Bacillus subtilis strain 34KLB
(SEQ ID NO: 1)
AGCTCGGATCCACTAGTAACGGCCGCCAGTGTGCTGGAATTCGCCCTTAG
AAAGGAGGTGATCCAGCCGCACCTTCCGATACGGCTACCTTGTTACGACT
TCACCCCAATCATCTGTCCCACCTTCGGCGGCTGGCTCCATAAAGGTTAC
CTCACCGACTTCGGGTGTTACAAACTCTCGTGGTGTGACGGGCGGTGTGT
ACAAGGCCCGGGAACGTATTCACCGCGGCATGCTGATCCGCGATTACTAG
CGATTCCAGCTTCACGCAGTCGAGTTGCAGACTGCGATCCGAACTGAGAA
CAGATTTGTGRGATTGGCTTAACCTCGCGGTTTCGCTGCCCTTTGTTCTG
TCCATTGTAGCACGTGTGTAGCCCAGGTCATAAGGGGCATGATGATTTGA
CGTCATCCCCACCTTCCTCCGGTTTGTCACCGGCAGTCACCTTAGAGTGC
CCAACTGAATGCTGGCAACTAAGATCAAGGGTTGCGCTCGTTGCGGGACT
TAACCCAACATCTCACGACACGAGCTGACGACAACCATGCACCACCTGTC
ACTCTGCCCCCGAAGGGGACGTCCTATCTCTAGGATTGTCAGAGGATGTC
AAGACCTGGTAAGGTTCTTCGCGTTGCTTCGAATTAAACCACATGCTCCA
CCGCTTGTGCGGGCCCCCGTCAATTCCTTTGAGTTTCAGTCTTGCGACCG
TACTCCCCAGGCGGAGTGCTTAATGCGTTAGCTGCAGCACTAAAGGGGCG
GAAACCCCCTAACACTTAGCACTCATCGTTTACGGCGTGGACTACCAGGG
TATCTAATCCTGTTCGCTCCCCACGCTTTCGCTCCTCAGCGTCAGTTACA
GACCAGAGAGTCGCCTTCGCCACTGGTGTTCCTCCACATCTCTACGCATT
TCACCGCTACACGTGGAATTCCACTCTCCTCTTCTGCACTCAAGTTCCCC
AGTTTCCAATGACCCTCCCCGGTTGAGCCGGGGGCTTTCACATCAGACTT
AAGAAACCGCCTGCGAGCCCTTTACGCCCAATAAtTCCGGACAACGCTTG
CCACCTACGTATTACCGCGGCTGCTGGCACGTAGTTAGCCGTGGCTTTCT
GGTTAGGTACCGTCAAGGTGCCGCCCTATTTGAACGGCACTTGTTCTTCC
CTAACAACAGAGCTTTACGATCCGAAAACCTTCATCACTCACGCGGCGTT
GCTCCGTCAGACTTTCGTCCATTGCGGAAGATTCCCTACTGCTGCCTCCC
GTAGGAGTCTGGGCCGTGTCTCAGTCCCAGTGTGGCCGATCACCCTCTCA
GGTCGGCTACGCATCGTCGCCTTGGTGAGCCGTTACCTCACCAACTAGCT
AATGCGCCGCGGGTCCATCTGTAAGTGGTAGCCGAAGCCACCTTTTATGT
CTGAACCATGCGGTTCAGACAACCATCCGGTATTAGCCCCGGTTTCCCGG
AGTTATCCCAGTCTTACAGGCAGGTTACCCACGTGTTACTCACCCGTCCG
CCGCTAACATCAGGGAGCAAGCTCCCATCTGTCCGCTCGACTTGCATGTA
TTAGGCACGCCGCCAGCGTTCGTCCTGAGCCATGAACAAACTCTAAGGGC
GAATTCTGCAGATATCCATCACACTGGCGGCCGCTCGAGCATGCATCTAG
AGGGCCCAATCGCCCTAT

In some embodiments, at least 15% by weight of the Bacillus bacterial species are Bacillus subtilis 34 KLB.

A first preferred Bacillus mixture includes 10% by weight of Bacillus licheniformis, 30% by weight of Bacillus pumilus, 30% by weight of Bacillus amyloliquefaciens, and 30% by weight of Bacillus mojavensis (referred to herein as Bacillus Mix #1).

A second preferred Bacillus mixture includes equal weights of Bacillus licheniformis, Bacillus pumilus, Bacillus amyloliquefaciens and Bacillus subtilis (referred to herein as Bacillus Mix #2). Preferably, at least two strains of Bacillus licheniformis and Bacillus subtilis are present in Bacillus Mix #2.

A third preferred Bacillus mixture includes Bacillus subtilis 34 KLB (Bacillus Mix #3).

The mixture of lactic-acid-producing bacterial species can include one to seven different strains of Bacteria. Exemplary lactic-acid-producing bacterial species include Pediococcus acidilactici, Pediococcus pentosaceus, Lactobacillus plantarum, or Bifidobacterium animalis. Preferably, the lactic-acid-producing bacterial species include Pediococcus acidilactici, Pediococcus pentosaceus, and Lactobacillus plantarum.

A preferred lactic-acid-producing mixture includes equal weights of Pediococcus acidilactici, Pediococcus pentosaceus, and Lactobacillus plantarum (referred to herein as Lactic Mix #1).

In some embodiments, the NPB composition of the present disclosure can include at least 94% by weight of a water soluble or water dispersible carbon source and about at least 0.1 to 1%, 0.1 to 2%, 0.1 to 3%, 0.1 to 4%, 0.1 to 5% by weight of Bacillus Mix #1 and/or of Bacillus Mix #2. Preferably, the NPB composition comprises about 0.1%, 0.2%, 0.3%, 0.4%, 0.5%, 0.6%, 0.7%, 0.8%, 0.9% or 1% by weight of Bacillus Mix #1 and/or of Bacillus Mix #2. In some embodiments, the NPB composition also includes about at least 0.1 to 1%, 0.1 to 2%, 0.1 to 3%, 0.1 to 4%, or 0.1 to 5% by weight of Bacillus Mix #3. Preferably, the NPB composition comprises about 0.1%, 0.2%, 0.3%, 0.4%, 0.5%, 0.6%, 0.7%, 0.8%, 0.9%, 1%, 2%, 3%, 4% or 5% by weight of Bacillus Mix #3.

In some embodiments, the NPB composition can include at least 94% by weight of a water soluble or water dispersible carbon source, about 0.1 to 1%, 0.1 to 2%, 0.1 to 3%, 0.1 to 4%, 0.1 to 5% by weight of Bacillus Mix #1, about 0.1 to 1%, 0.1 to 2%, 0.1 to 3%, 0.1 to 4%, 0.1 to 5% by weight of Bacillus Mix #2, and about 1 to 5% by weight of Lactic Mix #1. Preferably, the NPB composition comprises about 0.1%, 0.2%, 0.3%, 0.4%, 0.5%, 0.6%, 0.7%, 0.8%, 0.9%, or 1% by weight of Bacillus Mix #1, about 0.1 to 1%, 0.1 to 2%, 0.1 to 3%, 0.1 to 4%, 0.1 to 5% by weight of Bacillus Mix #2, and about 1%, 2%, 3% 4%, or 5% by weight of Lactic Mix #1.

In some embodiments, the NPB composition can include at least 94% by weight of a water soluble or water dispersible carbon source, about 0.1%, 0.2%, 0.3%, 0.4%, or 0.5% by weight of Bacillus Mix #1, about 0.1%, 0.2%, 0.3%, 0.4%, or 0.5% by weight of Bacillus Mix #2, about 1 to 5% by weight of Lactic Mix #1, and about 0.1%, 0.2%, 0.3%, 0.4%, 0.5% by weight of Bacillus Mix #3.

In some embodiments, the NPB composition can include equal weights of Bacillus Mix #1, Bacillus Mix #2, Bacillus Mix #3, and at least 4% by weight Lactic Mix #1.

In some embodiments, the NPB composition can include at least 0.4% by weight of Bacillus Mix #1, at least 0.4% by weight of Bacillus Mix #2, at least 0.4% by weight of Bacillus Mix #3, and at least 4% by weight of Lactic Mix #1.

In some embodiments, the mixture of Bacillus bacterial species in the NPB composition comprises Bacillus subtilis, Bacillus mojavensis, Bacillus licheniformis, Bacillus amyloliquefaciens, and Bacillus pumilus, where the Bacillus subtilis comprises Bacillus subtilis 34 KLB.

In some embodiments, the mixture of Bacillus bacterial species in the NPB composition comprises Bacillus subtilis, Bacillus subtilis 34 KLB, Bacillus licheniformis, Bacillus amyloliquefaciens, and Bacillus pumilus.

In some embodiments, the NPB composition includes equal amounts of Bacillus subtilis, Bacillus subtilis 34 KLB, Bacillus amyloliquefaciens, Bacillus licheniformis, and Bacillus pumilus by weight. In some embodiments, the NPB composition includes equal amounts of Lactobacillus plantarum, Pediococcus pentosaceus, and Pediococcus acidilactici by weight In some embodiments, the NPB composition includes equal amounts of Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici by weight. In some embodiments, the NPB composition includes about 1×10⁸ to 1×10¹¹ CFU/g of Bacillus subtilis, 1×10⁸ to 1×10¹¹ CFU/g of Bacillus subtilis 34 KLB, 1×10⁸ to 1×10¹¹ CFU/g of Bacillus amyloliquefaciens, 1×10⁸ to 1×10¹¹ CFU/g of Bacillus licheniformis, 1×10⁸ to 1×10¹¹ CFU/g of Bacillus pumilus, 1×10⁸ to 1×10¹¹ CFU/g of Lactobacillus plantarum, 1×10⁸ to 1×10¹¹ CFU/g of Pediococcus pentosaceus, and 1×10⁸ to 1×10¹¹ CFU/g of Pediococcus acidilactici.

Suitable water-soluble or water dispersible carbon sources include carbohydrates, proteins, polysaccharides, or mixtures thereof. In some embodiments, the water-soluble carbon source can include glucose, dextrose, fructose, erythrose, arabinose, ribose, deoxyribose, galactose, mannose, sucrose, lactose, maltose, dextrin, maltodextrin, glycerol, sorbitol, xylitol, inulin, trehalose, low molecular weight starches, modified starches, cellobiose, modified celluloses, amino acids, water soluble peptides, or mixtures thereof. In some embodiments, the dextrose is dextrose monohydrate. Suitable water-dispersible carbon sources include emulsified fats and oils. In some embodiments, the water-dispersible carbon source comprises soy lecithin, emulsified vegetable oil, or mixtures thereof. A preferred water-soluble or water dispersible carbon source is dextrose monohydrate.

In some embodiments, the NPB composition includes at least 50%, preferably at least 75%, and most preferably at least 90% by weight of a water-soluble or water-dispersible carbon source. In some embodiments, the NPB composition includes at least 91%, 92%, 93%, 94%, 95%, 96%, 97%, 98%, or 99% by weight of a water-soluble or water-dispersible carbon source. In some embodiments, the NPB composition includes 50%-99% by weight of a water-soluble or water-dispersible carbon source. In some embodiments, the NPB composition includes 60%-99% by weight of a water-soluble or water-dispersible carbon source. In some embodiments, the NPB composition includes 70%-99% by weight of a water-soluble or water-dispersible carbon source. In some embodiments, the NPB composition includes 75%-99% by weight of a water soluble or water dispersible carbon source. In some embodiments, the NPB composition includes 80%-99% by weight of a water soluble or water dispersible carbon source.

In other embodiments, the NPB composition can further include at least one inorganic mineral. The inorganic mineral can stimulate bacterial respiration and growth. Suitable inorganic minerals include disodium hydrogen phosphate, dipotassium hydrogen phosphate, sodium dihydrogen phosphate, potassium dihydrogen phosphate, sodium chloride, potassium chloride, magnesium sulfate, calcium sulfate, magnesium chloride, calcium chloride, and iron(III) chloride. The NPB composition comprises between 1 to 50%, 10 to 50%, 20 to 50%, 30 to 50%, or 40 to 50% by weight of the inorganic minerals. In some embodiments, the NPB composition comprises about 1%, 2%, 3%, 4%, 5%, 6%, 7%, 8%, 9%, 10%, 15%, 20%, 25%, 30%, 35%, 40%, 45%, or 50% by weight of the inorganic minerals.

In some embodiments, the NPB composition includes (a) between 75-99% w/w of anhydrous dextrose or dextrose monohydrate; (b) a mixture of Bacillus bacterial species comprising Bacillus subtilis, Bacillus mojavensis, Bacillus licheniformis, Bacillus amyloliquefaciens, and Bacillus pumilus, and wherein the Bacillus subtilis comprises Bacillus subtilis 34 KLB; and (c) a mixture of lactic-acid-producing bacterial species comprising Pediococcus acidilactici, Pediococcus pentosaceus, and Lactobacillus plantarum. In some embodiments, the mixture of Bacillus bacterial species has a bacterial concentration of at least 1×10⁶ colony forming units (CFU) per gram of the mixture, wherein each of the Bacillus species are individually fermented aerobically, dried and ground to an average particle size of about 200 microns. In some embodiments, the mixture of lactic-acid-producing bacterial species has a bacterial concentration of at least 1×10⁶ CFU per gram of the mixture, wherein each of the lactic-acid-producing species are fermented anaerobically, dried, and ground to an average particle size of about 200 microns.

In some embodiments, the NPB composition includes (a) between 75-95% w/w of anhydrous dextrose or dextrose monohydrate; (b) at least 1% w/w of a mixture containing Bacillus bacterial species comprising Bacillus subtilis, Bacillus mojavensis, Bacillus licheniformis, Bacillus amyloliquefaciens, and Bacillus pumilus, wherein each of the Bacillus species are individually fermented aerobically, dried and ground to an average particle size of about 200 microns, and wherein the Bacillus subtilis comprises Bacillus subtilis 34 KLB; and (c) at least 4% w/w of a mixture containing lactic-acid-producing bacterial species comprising Pediococcus acidilactici, Pediococcus pentosaceus, and Lactobacillus plantarum, wherein each of the lactic-acid-producing bacterial species are fermented anaerobically, dried, and ground to an average particle size of about 200 microns.

In some embodiments, the NPB composition includes about 98% dextrose monohydrate and equal amounts of Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici by weight. Each of the bacterial species can be individually fermented aerobically, dried, and ground.

Additional NPB compositions suitable for use in the compositions and methods of the present invention can include the compositions disclosed in U.S. Pat. No. 9,302,924 and WO2016070174, the contents of each of which are incorporated by reference in their entireties.

The CAD compositions can include one or more cyanuric acid reducing bacteria. In one aspect, the CAD composition includes Enterobacter cloacae, Bacillus subterraneous, a 16S organism, or a combination thereof. In some embodiments, the CAD composition further includes a biofilm forming bacteria. The biofilm forming bacteria can be, for example, Bacillus subtilis such as Bacillus subtilis 34 KLB.

In another aspect, the invention provides a biofilm containing the CAD compositions and a biofilm forming bacteria. In some embodiments, the biofilm includes Enterobacter cloacae and Bacillus subtilis 34 KLB. In some embodiments, the biofilm includes Bacillus subterraneous and Bacillus subtilis 34 KLB. In some embodiments, the biofilm includes a 16S organism and Bacillus subtilis 34 KLB. In some embodiments, the biofilm includes Enterobacter cloacae, Bacillus subterraneous, and Bacillus subtilis 34 KLB. In some embodiments, the biofilm includes Enterobacter cloacae, a 16S organism, and Bacillus subtilis 34 KLB. In some embodiments, the biofilm includes Bacillus subterraneous, a 16S organism, and Bacillus subtilis 34 KLB. In some embodiments, the biofilm includes Enterobacter cloacae, Bacillus subterraneous, a 16S organism, and Bacillus subtilis 34 KLB.

The biofilm can be on a solid support. In some embodiments, the biofilm can be produced when the cyanuric acid degrading bacteria is grown as a solid supported biofilm in combination with a known biofilm producing organism such as Bacillus subtilis 34 KLB.

In some embodiments, the method of the present disclosure involves spray coating a liquid slurry of the cyanuric acid degrading bacteria onto a solid support or filter medium.

The solid support or filter medium includes zeolite, wheat bran, rice bran, ground corn cobs, clays such as bentonite or kaolin, diatomaceous earth, activated charcoal, calcium carbonate, calcium pyrophosphate, tri-calcium phosphate, sphagnum moss, glass, sand, cellulose, ceramic, polyethylene, polypropylene, polystyrene, uncooked starch, or a mixture thereof. In some embodiments, the solid support is sphagnum moss.

In some aspects, the solid-supported bacteria is formulated as a tablet or a water-soluble packet.

In some aspects, the solid supported bacteria is enclosed in a porous filter housing. The porous filter housing has an average pore size in the range of 0.1 μm to 10.0 μm, e.g., 0.2 μm to 5.0 μm, 0.2 μm to 2.0 μm, or 0.1 μm to 1.0 μm. In some embodiments, the porous filter housing has an average pore size of about 1.0 μm. In some embodiments, the porous filter housing has an average pore size of about 0.8 μm. In some embodiments, the porous filter housing has an average pore size of about 0.6 μm. In some embodiments, the porous filter housing has an average pore size of about 0.5 μm. In some embodiments, the porous filter housing has an average pore size of about 0.4 μm.

In another aspect, the invention provides a filter assembly including the CAD composition dispersed on a solid support, where the solid support is enclosed in a porous filter housing.

In another aspect, the invention provides a filter assembly including a biofilm containing the CAD compositions dispersed on a solid support, where the solid support is enclosed in a porous filter housing. In some embodiments, the filter assembly includes a biofilm containing Enterobacter cloacae and Bacillus subtilis 34 KLB dispersed on a solid support, where the solid support is enclosed in a porous filter housing. In some embodiments, the filter assembly includes a biofilm containing Bacillus subterraneous and Bacillus subtilis 34 KLB dispersed on a solid support, where the solid support is enclosed in a porous filter housing. In some embodiments, the filter assembly includes a biofilm containing a 16S organism and Bacillus subtilis 34 KLB dispersed on a solid support, where the solid support is enclosed in a porous filter housing. In some embodiments, the filter assembly includes a biofilm containing Enterobacter cloacae, Bacillus subterraneous, and Bacillus subtilis 34 KLB dispersed on a solid support, where the solid support is enclosed in a porous filter housing. In some embodiments, the filter assembly includes a biofilm containing Enterobacter cloacae, a 16S organism, and Bacillus subtilis 34 KLB dispersed on a solid support, where the solid support is enclosed in a porous filter housing. In some embodiments, the filter assembly includes a biofilm containing a 16S organism, Bacillus subterraneous, and Bacillus subtilis 34 KLB dispersed on a solid support, where the solid support is enclosed in a porous filter housing. In some embodiments, the filter assembly includes a biofilm containing Enterobacter cloacae, a 16S organism, Bacillus subterraneous, and Bacillus subtilis 34 KLB dispersed on a solid support, where the solid support is enclosed in a porous filter housing.

The invention further includes a kit comprising one or more filter assemblies according to the invention and a NPB composition. In some embodiments, the filter assembly includes a biofilm containing Enterobacter cloacae and Bacillus subtilis 34 KLB dispersed on a solid support, where the solid support is enclosed in a porous filter housing. In some embodiments, the NPB composition includes greater than about 90% dextrose monohydrate and a mixture of Bacillus subtilis, Bacillus mojavensis, Bacillus amyloliquefaciens, Bacillus licheniformis, Bacillus pumilus, Pediococcus acidilactici, Pediococcus pentosaceus, and Lactobacillus plantarum.

In some embodiments, the NPB composition includes about 98% dextrose monohydrate and a mixture of Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici. In some embodiments, the Bacillus subtilis, Bacillus subtilis 34 KLB, Bacillus amyloliquefaciens, Bacillus licheniformis, Bacillus pumilus are present in equal amounts by weight. In some embodiments, the Lactobacillus plantarum, Pediococcus pentosaceus, and Pediococcus acidilactici are present in equal amounts by weight. In some embodiments, the Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici are present in equal amounts by weight.

The invention further includes methods of reducing the concentration of cyanuric acid in a recreational water system by dosing the water system with a NPB composition and placing the filter assembly of the invention in the water filter system. For example, the filter assembly is placed in the skimmer.

In some embodiments, the method of the invention includes: (a) dosing a pool with a composition comprising greater than about 90% by weight of dextrose monohydrate and a mixture of Bacillus subtilis, Bacillus mojavensis, Bacillus amyloliquefaciens, Bacillus licheniformis, Bacillus pumilus, Pediococcus acidilactici, Pediococcus pentosaceus, and Lactobacillus plantarum into the skimmer of a pool; and (b) placing, into the pool's skimmer, a second composition comprising a sphagnum moss supported biofilm comprising Enterobacter cloacae and Bacillus subtilis 34 KLB contained in a porous filter housing (e.g., a filter sock) with an average pore size less than about 1 micron. In some embodiments, Bacillus subtilis includes Bacillus subtilis 34 KLB.

In some embodiments, the method of the invention includes: (a) dosing a pool with a composition comprising greater than about 90% by weight of dextrose monohydrate and a mixture of Bacillus subtilis, Bacillus mojavensis, Bacillus amyloliquefaciens, Bacillus licheniformis, Bacillus pumilus, Pediococcus acidilactici, Pediococcus pentosaceus, and Lactobacillus plantarum into the skimmer of a pool; and (b) placing, into the pool's skimmer, a second composition comprising a sphagnum moss supported biofilm comprising Bacillus subterraneous and Bacillus subtilis 34 KLB contained in a porous filter housing (e.g., a filter sock) with an average pore size less than about 1 micron. In some embodiments, Bacillus subtilis includes Bacillus subtilis 34 KLB.

In some embodiments, the method of the invention includes: (a) dosing a pool with a composition comprising greater than about 90% by weight of dextrose monohydrate and a mixture of Bacillus subtilis, Bacillus mojavensis, Bacillus amyloliquefaciens, Bacillus licheniformis, Bacillus pumilus, Pediococcus acidilactici, Pediococcus pentosaceus, and Lactobacillus plantarum into the skimmer of a pool; and (b) placing, into the pool's skimmer, a second composition comprising a sphagnum moss supported biofilm comprising a 16S organism and Bacillus subtilis 34 KLB contained in a porous filter housing (e.g., a filter sock) with an average pore size less than about 1 micron. In some embodiments, Bacillus subtilis includes Bacillus subtilis 34 KLB.

In some embodiments, the method of the invention includes: (a) dosing a pool with a composition comprising greater than about 90% by weight of dextrose monohydrate and a mixture of Bacillus subtilis, Bacillus mojavensis, Bacillus amyloliquefaciens, Bacillus licheniformis, Bacillus pumilus, Pediococcus acidilactici, Pediococcus pentosaceus, and Lactobacillus plantarum into the skimmer of a pool; and (b) placing, into the pool's skimmer, a second composition comprising a sphagnum moss supported biofilm comprising Enterobacter cloacae, Bacillus subterraneous, and Bacillus subtilis 34 KLB contained in a porous filter housing (e.g., a filter sock) with an average pore size less than about 1 micron. In some embodiments, Bacillus subtilis includes Bacillus subtilis 34 KLB.

In some embodiments, the method of the invention includes: (a) dosing a pool with a composition comprising greater than about 90% by weight of dextrose monohydrate and a mixture of Bacillus subtilis, Bacillus mojavensis, Bacillus amyloliquefaciens, Bacillus licheniformis, Bacillus pumilus, Pediococcus acidilactici, Pediococcus pentosaceus, and Lactobacillus plantarum into the skimmer of a pool; and (b) placing, into the pool's skimmer, a second composition comprising a sphagnum moss supported biofilm comprising Enterobacter cloacae, a 16S organism, and Bacillus subtilis 34 KLB contained in a porous filter housing (e.g., a filter sock) with an average pore size less than about 1 micron. In some embodiments, Bacillus subtilis includes Bacillus subtilis 34 KLB.

In some embodiments, the method of the invention includes: (a) dosing a pool with a composition comprising greater than about 90% by weight of dextrose monohydrate and a mixture of Bacillus subtilis, Bacillus mojavensis, Bacillus amyloliquefaciens, Bacillus licheniformis, Bacillus pumilus, Pediococcus acidilactici, Pediococcus pentosaceus, and Lactobacillus plantarum into the skimmer of a pool; and (b) placing, into the pool's skimmer, a second composition comprising a sphagnum moss supported biofilm comprising a 16S organism, Bacillus subterraneous, and Bacillus subtilis 34 KLB contained in a porous filter housing (e.g., a filter sock) with an average pore size less than about 1 micron. In some embodiments, Bacillus subtilis includes Bacillus subtilis 34 KLB.

In some embodiments, the method of the invention includes: (a) dosing a pool with a composition comprising greater than about 90% by weight of dextrose monohydrate and a mixture of Bacillus subtilis, Bacillus mojavensis, Bacillus amyloliquefaciens, Bacillus licheniformis, Bacillus pumilus, Pediococcus acidilactici, Pediococcus pentosaceus, and Lactobacillus plantarum into the skimmer of a pool; and (b) placing, into the pool's skimmer, a second composition comprising a sphagnum moss supported biofilm comprising Enterobacter cloacae, Bacillus subterraneous, a 16S organism, and Bacillus subtilis 34 KLB contained in a porous filter housing (e.g., a filter sock) with an average pore size less than about 1 micron. In some embodiments, Bacillus subtilis includes Bacillus subtilis 34 KLB.

In some embodiments, the method of the invention includes: (a) dosing a pool with a composition comprising about 98% by weight of dextrose monohydrate and a mixture of Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici into the skimmer of a pool; and (b) placing, into the pool's skimmer, a second composition comprising a sphagnum moss supported biofilm comprising Enterobacter cloacae and Bacillus subtilis 34 KLB contained in a porous filter housing (e.g., a filter sock) with an average pore size less than about 1 micron. In some embodiments, Bacillus subtilis includes Bacillus subtilis 34 KLB. In some embodiments, the Bacillus subtilis, Bacillus subtilis 34 KLB, Bacillus amyloliquefaciens, Bacillus licheniformis, Bacillus pumilus are equal amounts by weight. In some embodiments, the Lactobacillus plantarum, Pediococcus pentosaceus, and Pediococcus acidilactici are equal amounts by weight. In some embodiments, the Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici are equal amounts by weight.

In some embodiments, the method of the invention includes: (a) dosing a pool with a composition comprising about 98% by weight of dextrose monohydrate and a mixture of Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici into the skimmer of a pool; and (b) placing, into the pool's skimmer, a second composition comprising a sphagnum moss supported biofilm comprising Bacillus subterraneous and Bacillus subtilis 34 KLB contained in a porous filter housing (e.g., a filter sock) with an average pore size less than about 1 micron. In some embodiments, Bacillus subtilis includes Bacillus subtilis 34 KLB. In some embodiments, the Bacillus subtilis, Bacillus subtilis 34 KLB, Bacillus amyloliquefaciens, Bacillus licheniformis, and Bacillus pumilus are equal amounts by weight. In some embodiments, the Lactobacillus plantarum, Pediococcus pentosaceus, and Pediococcus acidilactici are equal amounts by weight. In some embodiments, the Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici are equal amounts by weight.

In some embodiments, the method of the invention includes: (a) dosing a pool with a composition comprising about 98% by weight of dextrose monohydrate and a mixture of Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici into the skimmer of a pool; and (b) placing, into the pool's skimmer, a second composition comprising a sphagnum moss supported biofilm comprising a 16S organism and Bacillus subtilis 34 KLB contained in a porous filter housing (e.g., a filter sock) with an average pore size less than about 1 micron. In some embodiments, Bacillus subtilis includes Bacillus subtilis 34 KLB. In some embodiments, the Bacillus subtilis, Bacillus subtilis 34 KLB, Bacillus amyloliquefaciens, Bacillus licheniformis, Bacillus pumilus are equal amounts by weight. In some embodiments, the Lactobacillus plantarum, Pediococcus pentosaceus, and Pediococcus acidilactici are equal amounts by weight. In some embodiments, the Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici are equal amounts by weight.

In some embodiments, the method of the invention includes: (a) dosing a pool with a composition comprising about 98% by weight of dextrose monohydrate and a mixture of Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici into the skimmer of a pool; and (b) placing, into the pool's skimmer, a second composition comprising a sphagnum moss supported biofilm comprising Enterobacter cloacae, Bacillus subterraneous, and Bacillus subtilis 34 KLB contained in a porous filter housing (e.g., a filter sock) with an average pore size less than about 1 micron. In some embodiments, Bacillus subtilis includes Bacillus subtilis 34 KLB. In some embodiments, the Bacillus subtilis, Bacillus subtilis 34 KLB, Bacillus amyloliquefaciens, Bacillus licheniformis, Bacillus pumilus are equal amounts by weight. In some embodiments, the Lactobacillus plantarum, Pediococcus pentosaceus, and Pediococcus acidilactici are equal amounts by weight. In some embodiments, the Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici are equal amounts by weight.

In some embodiments, the method of the invention includes: (a) dosing a pool with a composition comprising about 98% by weight of dextrose monohydrate and a mixture of Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici into the skimmer of a pool; and (b) placing, into the pool's skimmer, a second composition comprising a sphagnum moss supported biofilm comprising Enterobacter cloacae, a 16S organism, and Bacillus subtilis 34 KLB contained in a porous filter housing (e.g., a filter sock) with an average pore size less than about 1 micron. In some embodiments, Bacillus subtilis includes Bacillus subtilis 34 KLB. In some embodiments, the Bacillus subtilis, Bacillus subtilis 34 KLB, Bacillus amyloliquefaciens, Bacillus licheniformis, Bacillus pumilus are equal amounts by weight. In some embodiments, the Lactobacillus plantarum, Pediococcus pentosaceus, and Pediococcus acidilactici are equal amounts by weight. In some embodiments, the Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici are equal amounts by weight.

In some embodiments, the method of the invention includes: (a) dosing a pool with a composition comprising about 98% by weight of dextrose monohydrate and a mixture of Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici into the skimmer of a pool; and (b) placing, into the pool's skimmer, a second composition comprising a sphagnum moss supported biofilm comprising a 16S organism, Bacillus subterraneous, and Bacillus subtilis 34 KLB contained in a porous filter housing (e.g., a filter sock) with an average pore size less than about 1 micron. In some embodiments, Bacillus subtilis includes Bacillus subtilis 34 KLB. In some embodiments, the Bacillus subtilis, Bacillus subtilis 34 KLB, Bacillus amyloliquefaciens, Bacillus licheniformis, Bacillus pumilus are equal amounts by weight. In some embodiments, the Lactobacillus plantarum, Pediococcus pentosaceus, and Pediococcus acidilactici are equal amounts by weight. In some embodiments, the Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici are equal amounts by weight.

In some embodiments, the method of the invention includes: (a) dosing a pool with a composition comprising about 98% by weight of dextrose monohydrate and a mixture of Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici into the skimmer of a pool; and (b) placing, into the pool's skimmer, a second composition comprising a sphagnum moss supported biofilm comprising Enterobacter cloacae, Bacillus subterraneous, a 16S organism, and Bacillus subtilis 34 KLB contained in a porous filter housing (e.g., a filter sock) with an average pore size less than about 1 micron. In some embodiments, Bacillus subtilis includes Bacillus subtilis 34 KLB. In some embodiments, the Bacillus subtilis, Bacillus subtilis 34 KLB, Bacillus amyloliquefaciens, Bacillus licheniformis, Bacillus pumilus are equal amounts by weight. In some embodiments, the Lactobacillus plantarum, Pediococcus pentosaceus, and Pediococcus acidilactici are equal amounts by weight. In some embodiments, the Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici are equal amounts by weight.

In some embodiments, the method of the invention includes: (a) dosing a first composition comprising greater than about 90% by weight of dextrose monohydrate and a mixture of Bacillus subtilis, Bacillus mojavensis, Bacillus amyloliquefaciens, Bacillus licheniformis, Bacillus pumilus, Pediococcus acidilactici, Pediococcus pentosaceus, and Lactobacillus plantarum into the skimmer of a pool; and (b) dosing a second composition comprising Bacillus subterraneous spray-coated onto a solid support into the pool's skimmer. In some embodiments, the solid-supported Bacillus subterraneous may be added into the skimmer directly as a free-flowing powder or placed in a dosing device with pore sizes sufficiently small to contain the powder but allow free flow of water through the device. In some embodiments, Bacillus subtilis includes Bacillus subtilis 34 KLB.

In some embodiments, the method of the invention includes: (a) dosing a first composition comprising greater than about 90% by weight of dextrose monohydrate and a mixture of Bacillus subtilis, Bacillus mojavensis, Bacillus amyloliquefaciens, Bacillus licheniformis, Bacillus pumilus, Pediococcus acidilactici, Pediococcus pentosaceus, and Lactobacillus plantarum into the skimmer of a pool; and (b) dosing a second composition comprising Enterobacter cloacae spray-coated onto a solid support into the pool's skimmer. In some embodiments, the solid-supported Enterobacter cloacae may be added into the skimmer directly as a free-flowing powder or placed in a dosing device with pore sizes sufficiently small to contain the powder but allow free flow of water through the device. In some embodiments, Bacillus subtilis includes Bacillus subtilis 34 KLB.

In some embodiments, the method of the invention includes: (a) dosing a first composition comprising greater than about 90% by weight of dextrose monohydrate and a mixture of Bacillus subtilis, Bacillus mojavensis, Bacillus amyloliquefaciens, Bacillus licheniformis, Bacillus pumilus, Pediococcus acidilactici, Pediococcus pentosaceus, and Lactobacillus plantarum into the skimmer of a pool; and (b) dosing a second composition comprising a 16S organism spray-coated onto a solid support into the pool's skimmer. In some embodiments, the solid-supported 16S organism may be added into the skimmer directly as a free-flowing powder or placed in a dosing device with pore sizes sufficiently small to contain the powder but allow free flow of water through the device. In some embodiments, Bacillus subtilis includes Bacillus subtilis 34 KLB.

In some embodiments, the method of the invention includes: (a) dosing a first composition comprising greater than about 90% by weight of dextrose monohydrate and a mixture of Bacillus subtilis, Bacillus mojavensis, Bacillus amyloliquefaciens, Bacillus licheniformis, Bacillus pumilus, Pediococcus acidilactici, Pediococcus pentosaceus, and Lactobacillus plantarum into the skimmer of a pool; and (b) dosing a second composition comprising Bacillus subterraneous and Enterobacter cloacae spray-coated onto a solid support into the pool's skimmer. In some embodiments, the solid-supported Bacillus subterraneous and Enterobacter cloacae may be added into the skimmer directly as a free-flowing powder or placed in a dosing device with pore sizes sufficiently small to contain the powder but allow free flow of water through the device. In some embodiments, Bacillus subtilis includes Bacillus subtilis 34 KLB.

In some embodiments, the method of the invention includes: (a) dosing a first composition comprising greater than about 90% by weight of dextrose monohydrate and a mixture of Bacillus subtilis, Bacillus mojavensis, Bacillus amyloliquefaciens, Bacillus licheniformis, Bacillus pumilus, Pediococcus acidilactici, Pediococcus pentosaceus, and Lactobacillus plantarum into the skimmer of a pool; and (b) dosing a second composition comprising Bacillus subterraneous and a 16S organism spray-coated onto a solid support into the pool's skimmer. In some embodiments, the solid-supported Bacillus subterraneous and 16S organism may be added into the skimmer directly as a free-flowing powder or placed in a dosing device with pore sizes sufficiently small to contain the powder but allow free flow of water through the device. In some embodiments, Bacillus subtilis includes Bacillus subtilis 34 KLB.

In some embodiments, the method of the invention includes: (a) dosing a first composition comprising greater than about 90% by weight of dextrose monohydrate and a mixture of Bacillus subtilis, Bacillus mojavensis, Bacillus amyloliquefaciens, Bacillus licheniformis, Bacillus pumilus, Pediococcus acidilactici, Pediococcus pentosaceus, and Lactobacillus plantarum into the skimmer of a pool; and (b) dosing a second composition comprising a 16S organism and Enterobacter cloacae spray-coated onto a solid support into the pool's skimmer. In some embodiments, the solid-supported 16S organism and Enterobacter cloacae may be added into the skimmer directly as a free-flowing powder or placed in a dosing device with pore sizes sufficiently small to contain the powder but allow free flow of water through the device. In some embodiments, Bacillus subtilis includes Bacillus subtilis 34 KLB.

In some embodiments, the method of the invention includes: (a) dosing a first composition comprising greater than about 90% by weight of dextrose monohydrate and a mixture of Bacillus subtilis, Bacillus mojavensis, Bacillus amyloliquefaciens, Bacillus licheniformis, Bacillus pumilus, Pediococcus acidilactici, Pediococcus pentosaceus, and Lactobacillus plantarum into the skimmer of a pool; and (b) dosing a second composition comprising Bacillus subterraneous, Enterobacter cloacae, and an 16S organism spray-coated onto a solid support into the pool's skimmer. In some embodiments, the solid-supported Bacillus subterraneous, Enterobacter cloacae, and 16S organism may be added into the skimmer directly as a free-flowing powder or placed in a dosing device with pore sizes sufficiently small to contain the powder but allow free flow of water through the device. In some embodiments, Bacillus subtilis includes Bacillus subtilis 34 KLB.

In some embodiments, the method of the invention includes: (a) dosing a first composition comprising about 98% by weight of dextrose monohydrate and a mixture of Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici into the skimmer of a pool; and (b) dosing a second composition comprising Bacillus subterraneous spray-coated onto a solid support into the pool's skimmer. In some embodiments, the solid-supported Bacillus subterraneous may be added into the skimmer directly as a free-flowing powder or placed in a dosing device with pore sizes sufficiently small to contain the powder but allow free flow of water through the device. In some embodiments, Bacillus subtilis includes Bacillus subtilis 34 KLB. In some embodiments, the Bacillus subtilis, Bacillus subtilis 34 KLB, Bacillus amyloliquefaciens, Bacillus licheniformis, and Bacillus pumilus are equal amounts by weight. In some embodiments, the Lactobacillus plantarum, Pediococcus pentosaceus, and Pediococcus acidilactici are equal amounts by weight. In some embodiments, the Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici are equal amounts by weight.

In some embodiments, the method of the invention includes: (a) dosing a first composition comprising about 98% by weight of dextrose monohydrate and a mixture of Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici into the skimmer of a pool; and (b) dosing a second composition comprising Enterobacter cloacae spray-coated onto a solid support into the pool's skimmer. In some embodiments, the solid-supported Enterobacter cloacae may be added into the skimmer directly as a free-flowing powder or placed in a dosing device with pore sizes sufficiently small to contain the powder but allow free flow of water through the device. In some embodiments, Bacillus subtilis includes Bacillus subtilis 34 KLB. In some embodiments, the Bacillus subtilis, Bacillus subtilis 34 KLB, Bacillus amyloliquefaciens, Bacillus licheniformis, and Bacillus pumilus are equal amounts by weight. In some embodiments, the Lactobacillus plantarum, Pediococcus pentosaceus, and Pediococcus acidilactici are equal amounts by weight. In some embodiments, the Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici are equal amounts by weight.

In some embodiments, the method of the invention includes: (a) dosing a first composition comprising about 98% by weight of dextrose monohydrate and a mixture of Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici into the skimmer of a pool; and (b) dosing a second composition comprising a 16S organism spray-coated onto a solid support into the pool's skimmer. In some embodiments, the solid-supported 16S organism may be added into the skimmer directly as a free-flowing powder or placed in a dosing device with pore sizes sufficiently small to contain the powder but allow free flow of water through the device. In some embodiments, Bacillus subtilis includes Bacillus subtilis 34 KLB. In some embodiments, the Bacillus subtilis, Bacillus subtilis 34 KLB, Bacillus amyloliquefaciens, Bacillus licheniformis, Bacillus pumilus are equal amounts by weight. In some embodiments, the Lactobacillus plantarum, Pediococcus pentosaceus, and Pediococcus acidilactici are equal amounts by weight. In some embodiments, the Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici are equal amounts by weight.

In some embodiments, the method of the invention includes: (a) dosing a first composition comprising about 98% by weight of dextrose monohydrate and a mixture of Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici into the skimmer of a pool; and (b) dosing a second composition comprising Bacillus subterraneous and Enterobacter cloacae spray-coated onto a solid support into the pool's skimmer. In some embodiments, the solid-supported Bacillus subterraneous and Enterobacter cloacae may be added into the skimmer directly as a free-flowing powder or placed in a dosing device with pore sizes sufficiently small to contain the powder but allow free flow of water through the device. In some embodiments, Bacillus subtilis includes Bacillus subtilis 34 KLB. In some embodiments, the Bacillus subtilis, Bacillus subtilis 34 KLB, Bacillus amyloliquefaciens, Bacillus licheniformis, and Bacillus pumilus are equal amounts by weight. In some embodiments, the Lactobacillus plantarum, Pediococcus pentosaceus, and Pediococcus acidilactici are equal amounts by weight. In some embodiments, the Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici are equal amounts by weight.

In some embodiments, the method of the invention includes: (a) dosing a first composition comprising about 98% by weight of dextrose monohydrate and a mixture of Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici into the skimmer of a pool; and (b) dosing a second composition comprising Bacillus subterraneous and a 16S organism spray-coated onto a solid support into the pool's skimmer. In some embodiments, the solid-supported Bacillus subterraneous and 16S organism may be added into the skimmer directly as a free-flowing powder or placed in a dosing device with pore sizes sufficiently small to contain the powder but allow free flow of water through the device. In some embodiments, Bacillus subtilis includes Bacillus subtilis 34 KLB. In some embodiments, the Bacillus subtilis, Bacillus subtilis 34 KLB, Bacillus amyloliquefaciens, Bacillus licheniformis, and Bacillus pumilus are equal amounts by weight. In some embodiments, the Lactobacillus plantarum, Pediococcus pentosaceus, and Pediococcus acidilactici are equal amounts by weight. In some embodiments, the Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici are equal amounts by weight.

In some embodiments, the method of the invention includes: (a) dosing a first composition comprising about 98% by weight of dextrose monohydrate and a mixture of Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici into the skimmer of a pool; and (b) dosing a second composition comprising 16S organism and Enterobacter cloacae spray-coated onto a solid support into the pool's skimmer. In some embodiments, the solid-supported 16S organism and Enterobacter cloacae may be added into the skimmer directly as a free-flowing powder or placed in a dosing device with pore sizes sufficiently small to contain the powder but allow free flow of water through the device. In some embodiments, Bacillus subtilis includes Bacillus subtilis 34 KLB. In some embodiments, the Bacillus subtilis, Bacillus subtilis 34 KLB, Bacillus amyloliquefaciens, Bacillus licheniformis, Bacillus pumilus are equal amounts by weight. In some embodiments, the Lactobacillus plantarum, Pediococcus pentosaceus, and Pediococcus acidilactici are equal amounts by weight. In some embodiments, the Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici are equal amounts by weight.

In some embodiments, the method of the invention includes: (a) dosing a first composition comprising about 98% by weight of dextrose monohydrate and a mixture of Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici into the skimmer of a pool; and (b) dosing a second composition comprising Bacillus subterraneous, Enterobacter cloacae, and a 16S organism spray-coated onto a solid support into the pool's skimmer. In some embodiments, the solid-supported Bacillus subterraneous, Enterobacter cloacae, and 16S organism may be added into the skimmer directly as a free-flowing powder or placed in a dosing device with pore sizes sufficiently small to contain the powder but allow free flow of water through the device. In some embodiments, Bacillus subtilis includes Bacillus subtilis 34 KLB. In some embodiments, the Bacillus subtilis, Bacillus subtilis 34 KLB, Bacillus amyloliquefaciens, Bacillus licheniformis, and Bacillus pumilus are equal amounts by weight. In some embodiments, the Lactobacillus plantarum, Pediococcus pentosaceus, and Pediococcus acidilactici are equal amounts by weight. In some embodiments, the Bacillus subtilis, Bacillus subtilis 34 KLB, Bacillus amyloliquefaciens, Bacillus licheniformis, Bacillus pumilus, Lactobacillus plantarum, Pediococcus pentosaceus, and Pediococcus acidilactici are equal amounts by weight.

In an alternative method, the NPB composition and the CAD composition are mixed together and added to a water-soluble pouch, which can then be dosed directly into the pool's skimmer. The water-soluble pouch may be comprised of any number of water-soluble films.

The methods of the invention can reduce the cyanuric acid concentration of the water system. For example, the cyanuric acid concentration can be reduced by at least 10%, at least 20%, at least 30%, at least 40%, at least 50%, at least 60%, at least 70%, at least 80%, at least 90%, 1 fold, 2 fold, 5 fold, 10 fold, or 20 fold as compared to the cyanuric acid concentration prior to the use of the methods disclosed herein. In some embodiments, the cyanuric acid concentration is reduced by at least 50% as compared to the cyanuric acid concentration prior to the use of the methods disclosed herein.

The bacteria are produced in such a way as to be fully water dispersible when added according to the method of the invention. Generally, each of the bacteria can be produced via submerged fermentation under conditions which optimize the growth of each organism. When the cell density of the fermentation reaches about 10¹¹-10¹² cfu/g, the individual bacteria are harvested via centrifugation and/or filtration, freeze dried to achieve a moisture level of about 5%, then ground to an average particle size of about 200 microns. The particle size can be measured using sieving according to ANSI/ASAE S319.4 method. The bacteria are then mixed in equal proportion and added to the carbon source of the instant composition. Typically, the final concentration of bacteria in the finished composition ranges from 10⁵ to 10¹² cfu/g, e.g., 10⁶ to 10¹¹ cfu/g, 10⁷ to 10¹¹ cfu/g, 10⁸ to 10¹¹ cfu/g, 10⁹ to 10¹¹ cfu/g, or 10⁹ to 10¹² cfu/g. The bacterial activity or bacterial concentration can be measured by traditional plate counting using agar, such as De Man, Rogosa and Sharpe (MRS) agar. For example, Bacillus counts can be obtained, for example, on Trypticase soy agar. Lactic acid counts can be obtained on MRS agar.

After fermentation, the bacteria can be harvested by any known methods in the art. For example, the bacteria are harvested by filtration or centrifugation, or simply supplied as the ferment. The bacteria can be dried by any method known in the art. For example, the bacteria can be dried by liquid nitrogen followed by lyophilization. The compositions according to the present disclosure are freeze dried to moisture content less than 20%, 15%, 10%, 9%, 8%, 7%, 6%, 5%, 4%, 3%, 2%, or 1% by weight. Preferably, the compositions according to the invention have been freeze dried to moisture content less than 5% by weight. In some embodiments, the freeze-dried powder is ground to decrease the particle size. The bacteria are ground by conical grinding at a temperature less than 10° C., 9° C., 8° C., 7° C., 6° C., 5° C., 4° C., 3° C., 2° C., 1° C., or 0° C. Preferably, the temperature is less than 4° C. For example, the particle size is less than 1500, 1400, 1300, 1200, 1100, 1000, 900, 800, 700, 600, 500, 400, 300, 200, or 100 microns. Preferably, the freeze-dried powder is ground to decrease the particle size such that the particle size is less than 800 microns. Most preferred are particle sizes less than about 400 microns. In most preferred embodiments, the dried powder has a mean particle size of 200 microns, with 60% of the mixture in the size range between 100-800 microns.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below.

All publications, patent applications, patents, and other references mentioned herein are expressly incorporated for reference in their entirety. In cases of conflict, the present specification, including definitions, will control. In addition, the material, methods, and examples described herein are illustrative only and are not intended to be limiting.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

### Definitions

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “comprising” as used herein is synonymous with “including” or “containing”, and is inclusive or open-ended and does not exclude additional, unrecited members, elements or method steps. By “consisting of is meant including, and limited to, whatever follows the phrase “consisting of.” Thus, the phrase “consisting of indicates that the listed elements are required or mandatory, and that no other elements may be present. By “consisting essentially of is meant including any elements listed after the phrase, and limited to other elements that do not interfere with or contribute to the activity or action specified in the disclosure for the listed elements. Thus, the phrase “consisting essentially of indicates that the listed elements are required or mandatory, but that other elements are optional and may or may not be present depending upon whether or not they materially affect the activity or action of the listed elements.

As used herein, the terms “bacteria” or “microbes” are used interchangeably to refer to micro-organisms that confer a benefit. The microbes according to the invention may be viable or non-viable. The non-viable microbes are metabolically-active. By “metabolically-active” as used herein is meant that they exhibit at least some respiration or residual enzyme, or secondary metabolite activity characteristic to that type of microbe.

By the term “non-viable” as used herein is meant a population of bacteria that is not capable of replicating under any known conditions. However, it is to be understood that due to normal biological variations in a population, a small percentage of the population (i.e. 5% or less) may still be viable and thus capable of respiration and/or replication under suitable growing conditions in a population which is otherwise defined as non-viable.

By the term “viable bacteria” as used herein is meant a population of bacteria that is capable of respiring and/or replicating under suitable conditions in which respiration and/or replication is possible. A population of bacteria that does not fulfill the definition of “non-viable” (as given above) is considered to be “viable.”

The term “recreational water system” as used herein is meant to include swimming pools, spas, hot tubs, jetted tubs or the like, and includes both salt water and fresh water systems.

The term “about” means within ±10% of a given value or range.

Unless stated otherwise, all percentages mentioned in this document are by weight based on the total weight of the composition.

A better understanding of the present invention may be given with the following examples which are set forth to illustrate, but are not to be construed to limit the present invention.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1

### Preparation of the Microbial Species

The microbial species of the present invention may be made by any of the standard fermentation processes known in the art. In the following examples, submerged liquid fermentation processes are described, however, where appropriate, solid state fermentation processes may be used.

Individual starter cultures of Bacillus subtilis, Bacillus amyloliquefaciens, Bacillus licheniformis, Bacillus pumilus, Bacillus subterraneous, and Enterobacter cloacae are grown according to the following general protocol: 2 grams nutrient broth, 2 grams yeast extract, and 4 grams maltodextrin were added to a 250 ml Erlenmeyer flask. One hundred milliliters distilled, deionized water was added and the flask stirred until all dry ingredients dissolved. The flask was then covered and placed for 30 minutes in an autoclave operating at 121° C., 15 psi. After cooling, the flask was inoculated with 1 ml of one of the pure microbial strains. The flask was sealed and placed on an orbital shaker at 30° C. Cultures were allowed to grow for 3-5 days. This process was repeated for each of the micro-organisms in the mixture. This process provided the starter cultures of each organism which were then used to prepare larger scale fermentations.

Individual Bacillus and Enterobacter fermentations were run under aerobic conditions at pH 7 and the temperature optimal for each species:

| Microbe | Temperature Optimum |
|---|---|
| Bacillus subtilis | 35° C. |
| Bacillus amyloliquefaciens | 30° C. |
| Bacillus licheniformis | 37° C. |
| Bacillus pumilus | 32° C. |
| Bacillus subterraneous | 30° C. |
| Enterobacter Cloacae | 37° C. |

After fermentation, the individual cultures were filtered, centrifuged, and freeze dried to a moisture level less than about 5%, then ground to a mean particle size of 200 microns with 60% of the product in a size range between 175-840 microns. The individual dried microbial cultures were then mixed in equal proportion by weight to obtain the microbial composition of the present invention. The final microbial concentration of the mixed powdered product is between 10⁹ and 10¹¹ CFU/g.

Individual, purified isolates of Pediococcus acidilactici, Pediococcus pentosaceus, and Lactobacillus plantarum were grown-up in separate fermenters using standard aerobic submerged liquid fermentation protocols. After fermentation, the individual cultures were filtered, centrifuged, and freeze dried to a moisture level less than about 5%, then ground to a mean particle size of 200 microns with 60% of the product in a size range between 175-840 microns. The individual dried microbial cultures were then mixed in equal proportion by weight to obtain the microbial composition of the present invention. The final microbial concentration of the mixed powdered product is between 10⁹ and 10¹¹ CFU/g.

### Example 2

### Formulation of Swimming Pool Treatment Products

The following formulations were prepared:

| | COMPOSITIONS | | | | |
|---|---|---|---|---|---|
| Ingredients | A | B | C | D | E |
| Bacillus subtilis | 1 × 10⁸ | | | | |
| | CFU/g | | | | |
| Bacillus subtilis 34 KLB | 1 × 10⁸ | 1 × 10⁸ | 1 × 10⁸ | | |
| | CFU/g | CFU/g | CFU/g | | |
| Bacillus | 1 × 10⁸ | | | | |
| amyloliquefaciens | CFU/g | | | | |
| Bacillus licheniformis | 1 × 10⁸ | | | | |
| | CFU/g | | | | |
| Bacillus pumilus | 1 × 10⁸ | | | | |
| | CFU/g | | | | |
| Bacillus subterraneous | | 1 × 10⁸ | | 1 × 10⁸ | 1 × 10⁸ |
| | | CFU/g | | CFU/g | CFU/g |
| Enterobacter cloacae | | | 1 × 10⁸ | | |
| | | | CFU/g | | |
| Lactobacillus plantarum | 1 × 10⁸ | | | | |
| | CFU/g | | | | |
| Pediococcus pentosaceus | 1 × 10⁸ | | | | |
| | CFU/g | | | | |
| Pediococcus acidilactici | 1 × 10⁸ | | | | |
| | CFU/g | | | | |
| Dextrose Monohydrate | 98.26% | | | | |
| Sphagnum Moss | | 100 | 100 | | |
| | | g | g | | |
| Zeolite A | | | | 98.0% | |
| | | | | by wt. | |
| Wheat Bran | | | | | 98.0% |
| | | | | | by wt. |

Compositions B and C were prepared according to the following protocol: A growth solution was prepared comprising 15 g/l Trypic soy broth, 2.0 g/l cyanuric acid, and 4 g/l dextrose and pH adjusted to 7.5. One hundred milliliters of the growth solution were aliquoted into 500 mL Erlenmeyer flasks. Sphagnum moss was added to the Erlenmeyer flask which was then capped and autoclaved at 121° C., 15 psi for 15 minutes. After cooling, the flasks were inoculated with 900 μL of a broth culture of the desired cyanuric acid degrading bacterium (E. cloacae or B. subterraneous) and 100 μL of a broth culture of Bacillus subtilis 34 KLB. The flasks were capped and incubated at 35° C. for 48 hours. After incubation, a visible biofilm was apparent on the sphagnum moss. This was aseptically retrieved from the Erlenmeyer flask and stored in sterile conditions until ready for use.

Compositions D and E were prepared by spray-coating a liquid suspension of Bacillus subterraneous onto the solid carrier.

### Example 3

### Identifying Cyanuric Acid Degrading Bacteria

Cyanuric acid screening medium was prepared as follows: 1.0 L of deionized water were decanted into a 2.0 L Erlenmeyer flask containing a Teflon coated stir bar. The flask was placed on a stirring hotplate set to maximum heat, 800 RPM. 0.25 g Calcium chloride, 0.124 g sodium bicarbonate, 0.3 g cyanuric acid, and 1.0 g of Composition A were added to the flask and allowed to fully dissolve. pH of the solution was adjusted to between 8.0-8.4 using dilute HCL and NaOH solutions as needed. This medium was then decanted in 250 mL aliquots into 500 mL Erlenmeyer flasks and capped securely with aluminum foil. The 500 mL flasks were autoclaved at 121° C., 15 psi for 15 minutes. Upon cooling, a flame-sterilized wire loop was used to transfer a colony of a suspected cyanuric acid degrading bacteria to the sterilized 500-mL cyanuric acid medium flasks. 25 mL of the medium was then extracted and placed into a 50-mL conical tube. The 50-mL conical tube was spun down in a centrifuge at 6000×g for 10 minutes. The supernatant was then pH adjusted to between 7.0-8.4 and the initial cyanuric acid level measured using an AquaCheck 7 test strip. The 500-mL inoculated cyanuric acid medium flasks were then capped and placed in an incubator/shaker set to 30° C., 200 RPM. Thereafter, 25 mL samples were extracted daily, following the above protocol, and assayed for residual cyanuric acid. Samples showing cyanuric acid reduction of at least 50% over a 5-day period were considered to be positive for cyanuric acid degradation and the corresponding bacterial strain(s) were taken through subsequent testing.

### Example 4

### Solid Substrate Reactor Flask Assays

A flame sterilized wire loop was used to transfer a colony of a cyanuric acid degrading bacteria as identified by the protocol in Example 3 to a flask containing sterile tryptic soy broth. The inoculated flasks were placed in an incubator/shaker set to 37° C., 200 RPM and allowed to incubate for 24 hours.

1.0 L of deionized water was decanted into a 2.0 L Erlenmeyer flask along with a stir bar. The flask was placed on a stirring hotplate set to maximum heat, 800 rpm. To this flask 0.25 g of calcium chloride, 0.124 g of sodium bicarbonate and 0.3 g of cyanuric acid. The mixture was heated and stirred until all the ingredients had dissolved then pH adjusted to between 8.0-8.4 using dilute HCl and NaOH as needed. This solution was then autoclaved at 121° C., 15 psi for 15 minutes. Upon cooling, 1.0 g of Composition A from Example 2 was added and allowed to fully dissolve.

Dry sphagnum moss (enough to cover the bottom of a culture dish) was added to a glass culture dish along with 50 mL of Tryptic Soy Broth. The dish was capped with aluminum foil and autoclaved at 121° C., 15 psi for 15 minutes. Upon cooling, another 100 mL of sterile tryptic soy broth was added to each culture dish and 25 mL of the cyanuric acid degrading bacteria culture in tryptic soy broth added. In some cases, 0.01 g of a 50×10⁹ cfu/g powder sample of B. subtilis 34 KLB was also added.

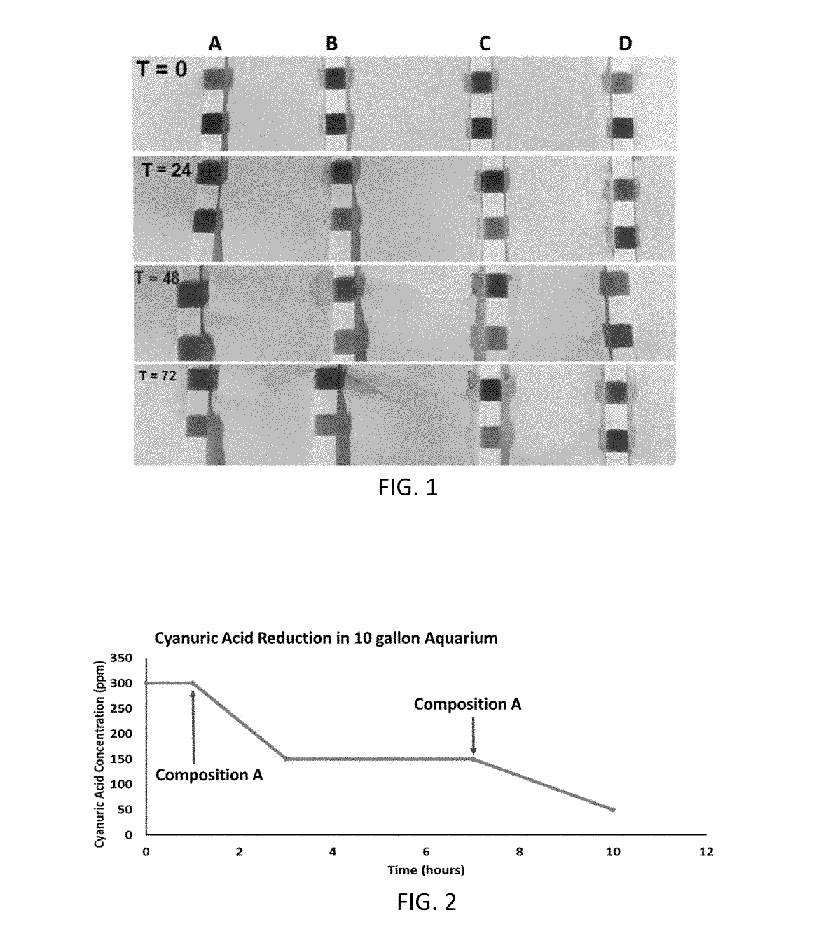

The inoculated sphagnum moss was aseptically transferred to a 0.5-micron sock filter. The sock filters containing the inoculated sphagnum moss were then placed inside a flask of cyanuric acid screening medium. As in Example 3, 25 mL of solution were extracted from the flasks and assayed for residual cyanuric acid level. Results are shown in FIG. 1. Both B. subterraneous and E. cloacae showed reductions of at least 50% of starting cyanuric acid level after 72 hours.

### Example 5

### Aquaria Assays

4.5 liters of saturated cyanuric acid solution were added to sterile, 10-gallon aquarium tanks. The tanks were then filled to capacity with sterile water and pH adjusted to 8.0-8.4. Sterilized AquaClear 110 filter systems were fitted to each tank. One (1) ppm free chlorine was added to each tank by addition of sodium hypochlorite. Free chlorine and total cyanuric acid levels were confirmed with the AquaCheck 7 test strips. Composition A from Example 2 was dosed into each aquarium at 10 mg/L.

Inoculated sphagnum moss samples (inoculated with Enterobacter cloacae) were prepared according to the method outlined in Example 4 and loaded into 0.5-micron filter socks. These were then placed in the filter housing of the AquaClear 110 filter system. Water samples were collected daily from each tank and tested for cyanuric acid levels. As needed, additional sodium hypochlorite was added daily to maintain a free chlorine level of 1 ppm.

FIG. 2 shows the synergy obtained by combining Composition A from Example 2 with sphagnum moss inoculated with E. cloacae (Composition C, Example 2). In the absence of Composition A, no cyanuric acid is reduced, however, 50% Cyanuric Acid removal is obtained within 5 hours of adding 10 mg/L Composition A to the tank. Each successive addition of Composition A yields an additional 50% Cyanuric acid reduction.

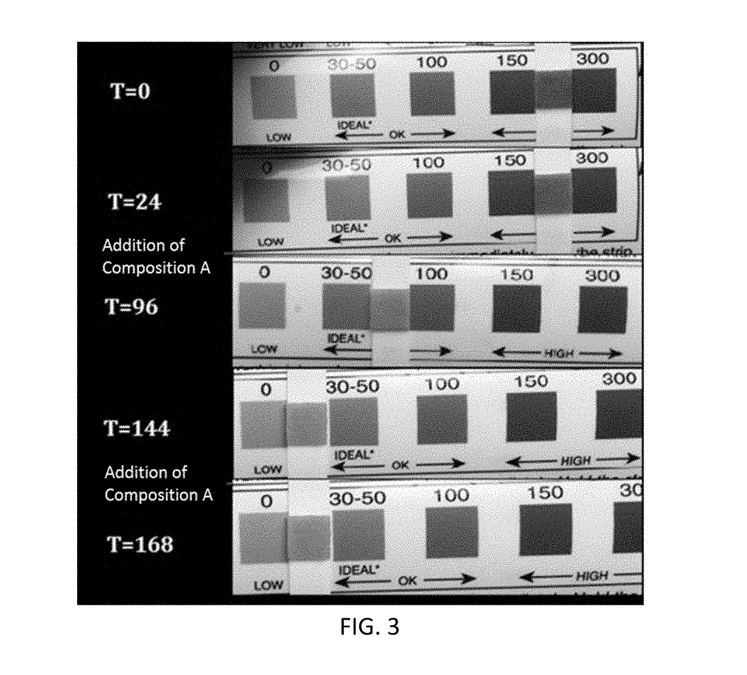

FIG. 3 shows the time course of the cyanuric acid removal using the AquaCheck 7 test strips.

### Example 6

### Flow Chart For Cyanuric Acid Reduction

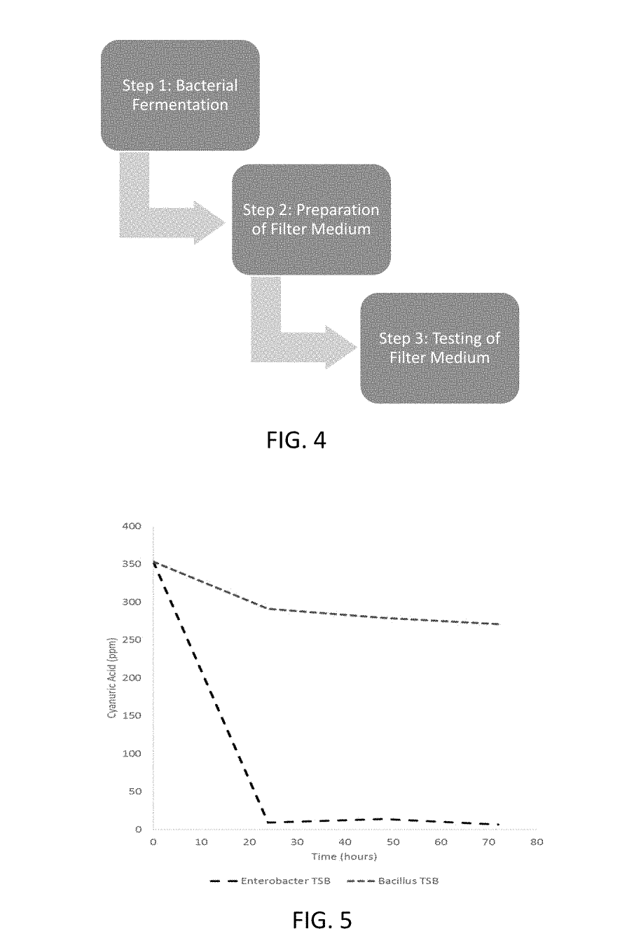

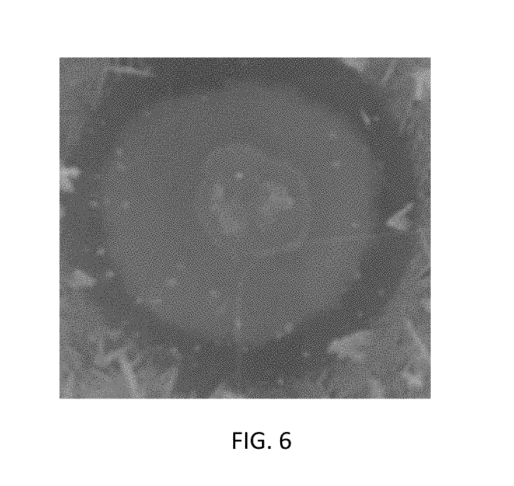

The flow chart is present in FIG. 4.

Step 1: Inducing Cyanuric Acid Amidohydrolase Activity in Bacterial Stocks.

Step (A) of step 1 includes: (i) prepare primary induction broth to begin the fermentation process. Composition is (per liter) Tryptic Soy Broth Powder (10.0 g), cyanuric acid (1.0 g), and dextrose (2.0 g) with pH adjusted to 7.5; (ii) dispense medium in 200 mL aliquots into sterile, capped Erlenmeyer flasks and autoclave at 121° C., 15 psi for 15 minutes. Allow to equilibrate to room temperature before inoculating; (iii) select a frozen glycerol stock of the desired bacterial strain. For each filter block, a minimum of two species are required: the biofilm-former and the cyanuric acid degrader. Using a flame-sterilized wire inoculating loop and following standard aseptic practices, inoculate a flask of primary induction broth with a loopful of frozen stock. Return the glycrol stock to cold storage; and (iv) place the flasks in an incubator/shaker set to 35° C., 150 RPM for 24 hours.

Step (B) of step 1 includes: (i) prepare secondary induction broth to continue the fermentation process. Composition is (per liter) Tryptic Soy Broth Powder (5.0 g), cyanuric acid (2.0 g), and dextrose (4.0 g) with pH adjusted to 7.5; (ii) dispense medium in 200 mL aliquots into sterile, capped Erlenmeyer flasks and autoclave at 121° C., 15 psi for 15 minutes. Allow to equilibrate to room temperature before inoculating; (iii) use 1.0mL of broth culture from the primary induction flasks to inoculate secondary induction flasks of the biofilm former and of the cyanuric acid degrader, respectively; and (iv) place the flasks in an incubator/shaker set to 35° C., 150 RPM for 24-48 hours.

Step 2: Preparing Biofilm-Seeded Filter Media.

Step 2 includes: (i) prepare biofilm growth broth with the following composition (per liter): Tryptic Soy Broth (15.0 g), cyanuric acid (2.0 g) and dextrose (4.0 g) pH adjusted to 7.5; (ii) dispense medium in 100mL aliquots into 500 mL Erlenmeyer flasks (one flask per filter medium treatment); (iii) cut a section of the desired filter media, sized to fit through the mouth of a 500 mL Erlenmeyer flask. Place it inside the flask of biofilm growth broth. The medium should come to rest half-covered in broth once inside the flask. Cap the flask containing the growth medium and the filter medium and autoclave at 121° C., 15 psi for 15 minutes. Allow to equilibrate to room temperature before inoculating; (iv) filter medium flasks will be inoculated with broth culture collected from the secondary induction flasks of each bacterium (which are 24-48 hours old). Using a micropipette (100-1,000 μL capacity) with sterile aerosol-barrier tips, inoculate the filter medium flask with 900 μL of the cyanuric acid degrading bacteria and 100 μL of the biofilm forming bacteria; (v) place inoculated flasks in a plate incubator and allow to sit undisturbed for 48 hours at 35° C.; and (vi) after 48 hours, a visible biofilm will have appeared across the broth surface. Bring the flask beneath a laminar flow hood and, using a pair of autoclaved forceps, grab the filter medium on the top portion and remove carefully from the flask. Taking care to avoid dripping, place the filter medium in a sterile sample bag and store for up to 4 hours in a cooler containing a cold pack before use.

Step 3: Testing Biofilm-Seeded Filter Medium for Efficacy

Step 3 includes: (i) prepare a concentrated cyanuric acid solution by dissolving 5.40 g of cyanuric acid in 2.0 L of hot tap water inside a 2.0 L Erlenmeyer flask. Use a stirrer/hotplate to stir and heat the solution until the cyanuric acid dissolves. Prepare a sufficient quantity to set up the desired number of 10-gallon tanks (each tank requires 4.2 L of concentrated cyanuric acid solution); (ii) clean each 10-gallon tank with 13% bleach and rinse well with tap water; (iii) add 4.2 liters of concentrated cyanuric acid solution to each 10-gallon tank. Fill the remaining volume with lukewarm tap water; (iv) to each tank, add 25 ppm dextrose (1.0 g per 10-gallon tank); (v) pH adjust each tank to 7.0-8.4 (to ensure accuracy of the AquaCheck7 Test Strips) using a sodium hydroxide solution. The amount of sodium hydroxide used will depend on the starting pH of the tap water; (vi) if desired, add free chlorine to the tank by adding 3 mL of 12.5% sodium hypochlorite to each tank (1-2 ppm free chlorine); (vii) hang a freshly-cleaned (with 13% bleach and well-rinsed with tap water) Fluval 20 filter on the outside of each tank. Into the filter basket, place the biofilm-seeded filter block (on the bottom of the basket) and a new, unseeded block (on top of the seeded one). Fill the filter with water from the tank and turn it on, with the speed adjusted to high power; (viii) take a T=0 cyanuric acid reading by testing with AquaCheck7 test strips according to manufacturer's instructions; (ix) after 24 hours, add 1.0 g (25 ppm) BiOWiSH Cyanuric Acid Reducer per 10-gallon tank. Take a T=24 cyanuric acid reading by testing with AquaCheck7 test strips according to manufacturer's instructions; and (x) allow the tanks to run for 96 hours before sampling again with AquaCheck7 strips. If desired, add 1.0 g (25 ppm) BiOWiSH Cyanuric Acid Reducer per 10-gallon tank and measure cyanuric acid again after 7 days.

### Equivalents

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

1. A filter assembly comprising:
a) a solid support;
b) a biofilm comprising:
(i) an organism having a 16S sequence at least 90% identical to SEQ ID NO: 2 and Bacillus subtilis 34 KLB;
(ii) Enterobacter cloacae and Bacillus subtilis 34 KLB,
(iii) Bacillus subterraneous and Bacillus subtilis 34 KLB, or
(iv) Bacillus subtilis 34 KLB and two or more organisms selected from Enterobacter cloacae, Bacillus subterraneous, and an organism having a 16S sequence at least 90% identical to SEQ ID NO: 2, wherein the biofilm is disposed on a solid support; and
c) a porous filter housing, wherein the solid support is encased in the porous filter housing.

2. A filter assembly comprising:
a) an organism having a 16S sequence at least 90% identical to SEQ ID NO: 2, Enterobacter cloacae, Bacillus subterraneous, or a combination thereof disposed on a solid support; and
b) a porous filter housing, wherein the solid support is encased in the porous filter housing.

3. The filter assembly of claim 1, wherein the solid support is porous.

4. The filter assembly of claim 1, wherein the solid support comprises zeolite, wheat bran, rice bran, ground corn cobs, bentonite, kaolin, diatomaceous earth, activated charcoal, calcium carbonate, calcium pyrophosphate, tri-calcium phosphate, sphagnum moss, glass, sand, cellulose, ceramic, polyethylene, polypropylene, polystyrene, uncooked starch, or a mixture thereof.

5. The filter assembly of claim 1, wherein the porous filter housing has an average pore size in the range of 0.2 μm to 1.0 μm.

6. The filter assembly of claim 5, wherein the porous filter housing has an average pore size of about 0.5 μm.

7. The filter assembly of claim 1, configured to be used in a swimming pool filtration system.

8. The filter assembly of claim 1, wherein the biofilm is in the form of a pellet or tablet.

9. A kit comprising the filter assembly of claim 1 and a bacterial composition comprising:
(a) a mixture of Bacillus bacterial species comprising Bacillus subtilis, Bacillus mojavensis, Bacillus licheniformis, Bacillus amyloliquefaciens, and Bacillus pumilus; and
(b) a mixture of lactic-acid-producing bacterial species comprising Pediococcus acidilactici, Pediococcus pentosaceus, and Lactobacillus plantarum.

10. The kit of claim 9, wherein the Bacillus subtilis comprises Bacillus subtilis 34 KLB.

11. A kit comprising the filter assembly of claim 1 and a bacterial composition comprising:
(a) a mixture of Bacillus bacterial species comprising Bacillus subtilis, Bacillus subtilis 34 KLB, Bacillus licheniformis, Bacillus amyloliquefaciens, and Bacillus pumilus; and
(b) a mixture of lactic-acid-producing bacterial species comprising Pediococcus acidilactici, Pediococcus pentosaceus, and Lactobacillus plantarum.

12. A method for reducing the concentration of cyanuric acid in a water system, comprising:
contacting the water system with the filter assembly of claim 1; and
contacting the water system with a bacterial composition, wherein the bacterial composition comprises:
(a) between 75-99% w/w of a water-soluble or water-dispersible carbon source;
(b) a mixture of Bacillus bacterial species comprising Bacillus subtilis, Bacillus subtilis 34 KLB, Bacillus licheniformis, Bacillus amyloliquefaciens, and Bacillus pumilus; and
(c) a mixture of lactic-acid-producing bacterial species comprising Pediococcus acidilactici, Pediococcus pentosaceus, and Lactobacillus plantarum.

13. A method for reducing the concentration of cyanuric acid in a water system, comprising:
contacting the water system with the filter assembly of claim 1; and
contacting the water system with a bacterial composition, wherein the bacterial composition comprises:
(a) between 75-99% w/w of a water-soluble or water-dispersible carbon source;
(b) a mixture of Bacillus bacterial species comprising Bacillus subtilis, Bacillus mojavensis, Bacillus licheniformis, Bacillus amyloliquefaciens, and Bacillus pumilus; and
(c) a mixture of lactic-acid-producing bacterial species comprising Pediococcus acidilactici, Pediococcus pentosaceus, and Lactobacillus plantarum.

14. The method of claim 12, wherein the water system is filtered through the filter assembly.

15. The method of claim 12, wherein the bacterial species in the bacterial composition are non-pathogenic.

16. The method of claim 12, wherein at least 15% of the Bacillus bacterial species in the bacterial composition are Bacillus subtilis 34 KLB.

17. The method of claim 12, wherein each of the lactic-acid-producing bacterial species in the bacterial composition are present in equal amounts by weight.

18. The method of claim 12, wherein the bacterial composition further comprises an inorganic mineral that stimulates bacterial respiration and growth.

19. The method of claim 18, wherein the inorganic mineral is selected from the group consisting of disodium hydrogen phosphate, dipotassium hydrogen phosphate, sodium dihydrogen phosphate, potassium dihydrogen phosphate, sodium chloride, potassium chloride, magnesium sulfate, calcium sulfate, magnesium chloride, calcium chloride, and iron(III) chloride.

20. The method of claim 12, further comprising placing the filter assembly into a filtration system in connection with the water system.

21. The method of claim 12, wherein the water system is a swimming pool.

22. The method of claim 12, wherein the water-soluble or water-dispersible carbon source is selected from the group consisting of acetate, succinate, dextrose, sucrose, fructose, erythrose, arabinose, ribose, deoxyribose, galactose, mannose, lactose, maltose, dextrin, maltodextrin, glycerol, sorbitol, xylitol, inulin, trehalose, starch, cellobiose, and carboxy methyl cellulose.

23. The method of claim 22, wherein the dextrose is dextrose monohydrate.

24. The method of claim 12, wherein the bacterial composition has a bacterial concentration of about 0.01 to 10 ppm.

25. The method of claim 12, wherein the biofilm of the filter assembly has a bacterial concentration of about 0.01 to 10 ppm.

26. The method of claim 12, wherein the Bacillus subtilis comprises Bacillus subtilis 34 KLB.

27. The method of claim 12, wherein the mixture of Bacillus bacterial species has a bacterial concentration of at least 1×10⁶ colony forming units (CFU) per gram of the mixture, wherein each of the Bacillus species are individually fermented aerobically, dried, and ground to an average particle size of about 200 microns.

28. The method of claim 12, wherein the mixture of lactic-acid-producing bacterial species has a bacterial concentration of at least 1×10⁶ colony forming units (CFU) per gram of the mixture, wherein each of the lactic-acid-producing species are fermented anaerobically, dried, and ground to an average particle size of about 200 microns.

29. The method of claim 12, wherein the concentration of cyanuric acid in the water system can be reduced by at least 10%.

30. (canceled)
